(19) United States
(12) Patent Application Publication
(10) Pub. No.: US 2010/0152052 A1
(43) Pub. Date: Jun. 17, 2010

(54) SPECIMENS FOR MICROANALYSIS PROCESSES

(76) Inventors: Steven L. Goodman, Madison, WI (US); Thomas F. Kelly, Madison, WI (US); Terri J. Tomicki, Janesville, WI (US)

Correspondence Address: PERKINS COIE LLP;PATENT-SEA, P.O. BOX 1247, SEATTLE, WA, 98111-1247, US

(21) Appl. No.: 11/997,141

(22) PCT Filed: Jul. 28, 2006
(86) PCT No.: PCT/US2006/029323

§ 371 Date: Jul. 11, 2008

Related U.S. Application Data
(60) Provisional application No. 60/703,096, filed on Jul. 28, 2005.

(51) Int. Cl. C40B 30/00; C12Q 1/70; C12Q 1/02; G01N 1/00; C40B 50/14

(52) U.S. Cl. 506/7; 435/5; 435/29; 436/176; 506/30

(57) ABSTRACT

The present invention relates to specimens for use in microanalysis processes. One aspect of the invention is directed toward using a mold to form specimens for a microanalysis process (e.g., including an atom probe and/or transmission electron microscope processes). Other aspects of the invention are directed towards embedding specimen material (e.g., including nanoparticles) in an embedment material to produce a specimen suitable for use in a microanalysis process. Still other aspects include combining specimen material with an embedment material to enhance a microanalysis process. Yet other embodiments of the invention are directed toward combining a specimen material with multiple embedment materials to produce specimens suitable for a microanalysis process. Further aspects of the invention are directed toward analyzing at least a portion of a specimen produced by one or more of the processes discussed above.

### CROSS-REFERENCE TO RELATED APPLICATION(S)

This application claims the benefit of U.S. Provisional Patent Application No. 60/703,096, filed Jul. 28, 2005, entitled ATOM PROBE SPECIMENS, which is fully incorporated herein by reference.

### TECHNICAL FIELD

Embodiments of the present invention relate to specimens for use in microanalysis processes, including specimens created via a casting process and/or atom probe specimens.

### BACKGROUND

Nanoparticles of various types and compositions are finding increasing applications in biomedicine for functions as diverse as detectors, optical and electron microscope labels, contrast agents for diagnostic magnetic resonance and optical coherence tomography imaging, bio-separations, catalysis, and drug delivery devices. Nanoparticulate materials are also extremely important in many non-medical applications including catalysis, material coatings, data storage, nano-electronics, cosmetics (e.g., sunscreen), and many other applications in order to impart unique properties to these various materials and devices. For example, nanoparticles can include magnetic and paramagnetic particles, metal colloids, semiconductor quantum dots, carbon nanotubes and nanowires, metal oxides, organic particles, fullerenes, biological particles and macromolecular complexes (proteins, viruses, and ribosomes), various types of colloids, nanoshells, dendrimers, and the like.

The special properties of nanoparticles that have created such excitement in the biomedical, biotechnology, and nanotechnology communities are due to their quantum-level properties. By one commonly used definition, nanoparticles are no larger than 100 nm in size; therefore each individual particle consists of a small, finite number of atoms. For example, a 4 nm diameter nanoparticle contains only about 4000 atoms. Because nanoparticles are often composed of only a few atoms, the position and type of each individual atom can be important. Therefore, in order to develop better nanoparticles and improve or develop nanoparticle-based devices and technologies, it is imperative to understand their structure at the atomic level. Unfortunately, nanoparticles can be difficult to analyze and are often do not have a size, shape, and geometry that is suitable for many microanalysis processes.

### SUMMARY

The present invention is directed generally toward specimens for use in microanalysis processes. One aspect of the invention is directed toward a method for producing a specimen for a microanalysis processes that includes providing specimen material to be analyzed via a microanalysis process and placing the specimen material into a mold configured to form the specimen material into a shape suitable for the microanalysis process. The method further includes forming a specimen suitable for use in the microanalysis process using the mold. The specimen includes the specimen material. A further aspect of the invention is directed toward analyzing at least a portion of the specimen produced by the method discussed above using the microanalysis process.

Other aspects of the invention are directed toward a method for producing a specimen for a microanalysis processes that includes providing a specimen material to be analyzed via a microanalysis process, providing an embedment material, and binding the specimen material and the embedment material together. The specimen material includes multiple noncontiguous portions spaced apart from one another in the embedment material. The method further includes forming a specimen from the specimen material and the embedment material that are bound together. The specimen includes the multiple noncontiguous portions spaced apart from one another in the embedment material. A further aspect of the invention is directed toward analyzing at least a portion of the specimen produced by the method discussed above using the microanalysis process.

Still other aspects of the invention are directed toward a method for producing a specimen for a microanalysis processes that includes providing a specimen material to be analyzed via a microanalysis process, providing an embedment material, and binding the specimen material and the embedment material together. The embedment material has a selected thermal and/or electrical conductivity characteristic. The method further includes forming a specimen from the specimen material and the embedment material that are bound together. A further aspect of the invention is directed toward analyzing at least a portion of the specimen produced by the method discussed above using the microanalysis process.

Yet other aspects of the invention are directed toward a method for producing a specimen for a microanalysis processes that includes providing a specimen material to be analyzed via a microanalysis process, providing a first embedment material, and binding the specimen material and the first embedment material together. The method further includes providing a second embedment material and binding the second embedment material to a portion of the specimen material and/or a portion of the second embedment material. The method still further includes forming a specimen from the specimen material, the first embedment material, and the second embedment material after the second embedment material is bound to the portion of the specimen material and/or the portion of the second embedment material. A further aspect of the invention is directed toward analyzing at least a portion of the specimen produced by the method discussed above using the microanalysis process.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is a partially schematic illustration of a microanalysis device analyzing a specimen on a micro level (e.g., on a near molecular level, near atomic level, or elemental level) in accordance with selected embodiments of the invention.

FIG. 2 is a flow diagram illustrating a process for producing a specimen and/or analyzing a specimen material in accordance with certain embodiments of the invention.

FIG. 3 is a partially schematic side view illustration of a microtip array having a shape that is suitable for analysis in an AP in accordance with selected embodiments of the invention.

FIG. 4 is a partially schematic top view illustration of the microtip array shown in FIG. 3.

FIG. 5 is a partially schematic illustration of the microtip array shown in FIG. 3 pressed into a mold material in accordance with certain embodiments of the invention.

FIG. 6 is a partially schematic illustration of a mold formed in the mold material after the microtip array shown in FIG. 5 has been removed in accordance with selected embodiments of the invention.

FIG. 7 is a partially schematic illustration of a processing arrangement in accordance with certain embodiments of the invention.

FIG. 8 is a partially schematic illustration of a mold being filled with a specimen material in accordance with selected embodiments of the invention.

FIG. 9 is a partially schematic illustration of the specimen material being positioned in the mold (shown in FIG. 8) by a plunger in accordance with certain embodiments of the invention.

FIG. 10 is a partially schematic illustration of a specimen after it has been removed from the mold shown in FIGS. 8 and 9 using the plunger in accordance with selected embodiments of the invention.

FIG. 11 is a partially schematic illustration of the specimen material being positioned in the mold by a plunger in accordance with other embodiments of the invention.

FIG. 12 is a partially schematic illustration of a specimen after it has been removed from the mold shown in FIG. 11 using the plunger in accordance with other embodiments of the invention.

FIG. 13 is a partially schematic illustration of a mold being filled with a single piece of specimen material in accordance with selected embodiments of the invention.

FIG. 14 is a partially schematic illustration of a specimen suitable for an atom probe process prior to initiating atom probe analysis in accordance with certain embodiments of the invention.

FIG. 15 is a partially schematic illustration of the specimen shown in FIG. 14 after atom probe analysis has been initiated in accordance with selected embodiments of the invention.

FIG. 16 is a partially schematic illustration of the specimen shown in FIG. 15 after atom probe analysis has been continued in accordance with certain embodiments of the invention.

FIG. 17 is a table illustrating field evaporation characteristics for certain types of materials in accordance with selected embodiments of the invention.

FIG. 18 is a table illustrating information on other materials in accordance with certain embodiments of the invention.

FIG. 19 is a partially schematic front view illustration of a wedge shaped specimen in accordance with selected embodiments of the invention.

FIG. 20 is a partially schematic side view illustration of the specimen shown in FIG. 19.

FIG. 21 is a partially schematic front view illustration of specimen including a first part suitable for a first microanalysis process and a second part suitable for a second microanalysis process in accordance with certain embodiments of the invention.

FIG. 22 is a partially schematic side view illustration of the specimen shown in FIG. 21.

FIG. 23 is a flow diagram illustrating a process for producing a specimen and/or analyzing a specimen material in accordance with selected embodiments of the invention.

FIG. 24 is a flow diagram illustrating a process for producing a specimen and/or analyzing a specimen material in accordance with other embodiments of the invention.

FIG. 25 is a partially schematic cross-sectional view of a specimen that includes a specimen material, a first embedment material, and a second embedment material in accordance with certain embodiments of the invention.

### DETAILED DESCRIPTION

In the following description, numerous specific details are provided in order to give a thorough understanding of embodiments of the invention. One skilled in the relevant art will recognize, however, that the invention may be practiced without one or more of the specific details, or with other methods, components, materials, etc. In other instances, well known structures, materials, or operations are not shown or described in order to avoid obscuring aspects of the invention.

References throughout the specification to “one embodiment” or “an embodiment” means that a particular feature, structure, or characteristic described in connection with the embodiment is included in at least one embodiment of the present invention. Thus, the appearances of the phrase “in one embodiment” or “in an embodiment” in various places throughout the specification are not necessarily all referring to the same embodiment. Furthermore, the particular features, structures, or characteristics may be combined in any suitable manner in one or more embodiments. Additionally, as used herein, casting is a process by which a material is introduced into a mold, shaped, and then removed producing a fabricated object or part. For example, in selected embodiments a liquid, mixture, suspension, or the like can be introduced into a mold and solidified. In other embodiments, one or more pieces of solid material can be placed in a mold and pressure applied to form the fabricated object (e.g., by applying pressure, sintering, or the like). Furthermore, herein the finished product of a casting process is called a casting or a cast object (e.g., a cast specimen).

Various embodiments discussed below provide a method for producing a specimen for a microanalysis process and/or a method for analyzing a specimen material. For example, selected embodiments are directed toward methods for forming a specimen suitable for use in a microanalysis process. In some embodiments, specimens that do not have the desired shape, size, and/or geometry can be formed or cast into a form suitable for analysis. In certain embodiments, an embedment material having a selected characteristic can be combined with specimen material (e.g., the material of interest) to form a specimen that will have a certain characteristic during microanalysis.

In selected embodiments, methods described below can be used to perform structural and compositional analysis of nanoparticulate and micro-particulate materials, whether these particulate are of natural, biological or synthetic (man-made) origin. Such particulates may be inorganic, organic or composed of a combination of inorganic and organic materials. For example, specimens that can be examined via these methods can include (without limitation), biological materials such as proteins, nucleic acids, biomacromolecules, biomacromolecular complexes and viruses, organic nano-particles (e.g., dendrimers, polymers, fullerenes, and the like), inorganic nano-particles (e.g., ceramics, dielectrics, colloids, and micro- and nano-particulate materials), and nano-porous and micro-porous catalysts, zeolites, and other materials having nano-scale or micro-scale voids and cavities.

In certain embodiments, the specimen material may not be nanoparticulate in its present, original, or native state. Accordingly, the specimen material can be prepared by breaking down/separating the specimen material into multiple portions (e.g., small particulates). In some embodiments, these materials can be extremely small. For example, in selected embodiments the specimen material can be processed into particles or portions with at least two dimensions less than about 1 micron. For example, in selected embodiments the specimen material can be cut, diced, ground, pulverized, fractured, or the like.

In other embodiments, the specimen material can be processed into particles or portions even smaller. For example, in certain embodiments the specimen material can be processed into portions that are on the molecular or atomic level. For example, in certain embodiments the specimen material can be melted (e.g., and placed in a mold to cast a specimen). In still other embodiments, the specimen material can be dissolved in another medium or material. For example, in selected embodiments the specimen material can be dissolved in a solvent and the solvent can then be evaporated to form a specimen in a mold or a structure of specimen material. In another embodiment, the specimen material can be dissolved into an embedment material and a specimen can be formed that includes both the specimen material and the embedment material. In still other embodiments, the specimen material can be dissolved into another medium by transforming the specimen material into a gaseous state and bubbling the gas through a liquid solvent or embedment material to combine the specimen material with the solvent or embedment material. The combined materials in liquid form can then be placed in a mold to cast a specimen or the liquid can be transformed into another type of solid structure and a specimen can be formed from the structure.

In still other embodiments, methods described below can be used to perform structural and compositional analysis of organic and/or inorganic particulate or nanoparticulate materials such as fullerenes, ceramics, dielectrics, nano- and micro-porous catalysts, zeolites, colloids, and other micro and nano-particulate materials. These materials can include magnetic and paramagnetic particles, metal colloids, semiconductor quantum dots, nanowires, metal oxides, organic particles, fullerenes, biological particles and macromolecular complexes (proteins, viruses, and ribosomes), various types of colloids, nanoshells, dendrimers, and the like. In yet other embodiments, methods described below can be used to perform structural and compositional analysis of biological and organic materials, including (without limitation) proteins, lipids, carbohydrates, and nucleic acids, as well as biomolecular and biomacromolecular assemblies such as receptor complexes, receptors coupled with ligands, enzyme-substrate complexes, drug-target complexes, membranes, membrane-bound proteins, cellular organelles, viruses, and portions of whole cells and other biological components, including tissue specimens, proteins, polynucleic acids, oligonucleotides, macromolecular complexes or other structures that are located within biological tissues, cellular components, cellular organelles, extracellular organelles, viruses, bacteria, other micro-organisms, or other biological systems or components. In still other embodiments, methods described below can be used to perform structural and compositional analysis of man-made or partially man-made biological structures, including tissue engineering scaffolds, cell culture systems, and other biological-synthetic constructs.

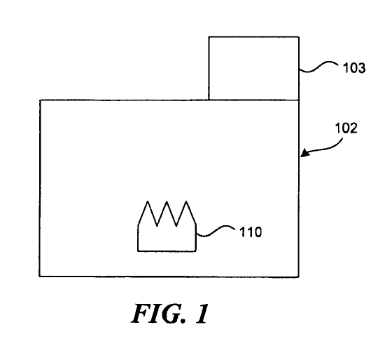

FIG. 1 is a partially schematic illustration of a microanalysis device 102 analyzing a specimen 110 on a micro level (e.g., near molecular level, near atomic level, or elemental level) in accordance with selected embodiments of the invention. For example, the microanalysis device 102 in FIG. 1 can include a Scanning Probe Microscope (“SPM”), Scanning Electron Microscope (“SEM”), a Transmission Electron Microscope (“TEM”), an Atomic Force Microscope (“AFM”), a Matrix-Assisted Laser Desorption/ionization (“MALDI”) instrument, a Secondary Ion Mass Spectrometer (“SIMS”), a Particle-Induced X-Ray Emission (“PIXE”) device, an Energy-Dispersive Spectroscope (“EDS”), an X-Ray Fluorescence Spectroscope (“XRF”), a diffraction process (e.g., process including light, photons, X-Rays, neutrons, or the like), an Atom Probe (“AP”) or other mass spectrometer processes, or the like. In certain embodiments, the microanalysis device 102 can include a computing system 103 to run at least a portion of the microanalysis process and/or to process data. In other embodiments, the computing system 103 can be distributed and/or separate from the microanalysis device 102.

For example, a three-dimensional atom probe (“AP”) is an analytical instrument capable of providing atomic-scale three-dimensional compositional data. Various embodiments of an atom probe include an ultra high vacuum (“UHV”) chamber in which a very sharp needle-shaped specimen is placed facing a detector that encodes in two dimensions. A large DC potential (e.g., 5 kV) is placed on the specimen that is almost, but not quite, sufficient to field ionize the specimen atoms on the apex. A very fast excitation pulse (e.g., an energy pulse at a pulse rate of up to several hundred kilohertz) is applied to the specimen or a counter electrode. The magnitude of the pulse is chosen such that the combined magnitude of the DC potential and excitation pulse energy is sufficient to occasionally (e.g., one time in 100 pulses) ionize a single atom near the tip of the specimen. This process is called field evaporation (“FE”).

The evaporated ion is accelerated away from the specimen and strikes a detector that records the location of the impact. The time required for the ion to fly from the specimen to the detector (e.g., the Time of Flight [“TOF”]) is related to the ion's mass-to-charge ratio. Consequently, the elemental identity of each ion can be determined from its TOF. Additionally, the location at which the ion hits the detector and the order in which the ion arrives at the detector can be correlated to its original position on the apex of the specimen. Combining the TOF data with the two-dimensional detector information allows the atomic composition of the specimen to be determined in three dimensions.

The excitation pulse(s) can include various forms of energy and can include varying pulse rates. For example, in certain embodiments the excitation pulse(s) can include one or more of the following: a voltage pulse, an electron beam or packet, an ion beam, a laser pulse (e.g., as used in a Pulsed Laser Atom Probe [“PLAP“]), or some other suitable pulsed source. An example of a suitable AP is a Local Electrode Atom Probe (“LEAP®”) available from Imago Scientific Instruments Corporation of Madison, Wis. Although for the purpose of illustration, many of the following embodiments are discussed with reference to laser and/or voltage pulsed atom probes, one skilled in the art will understand that the underlying principles are equally applicable to a wide variety of pulse excitation source(s).

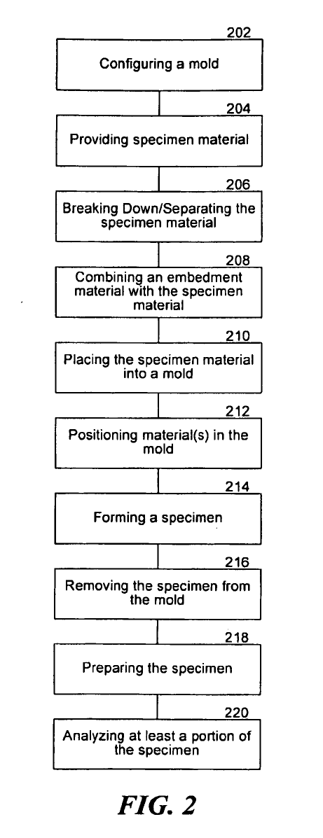

In many microanalysis processes the size, shape, and geometry of the specimen can greatly affect the quality of the analysis process. For example, in an AP, the specimen is the imaging optic, therefore specimen preparation can be extremely important for obtaining useful data. In selected embodiments, the specimen radius can effectively determine the image magnification and the field of view in the AP. FIG. 2 is a flow diagram illustrating a process for producing a specimen (e.g., for a microanalysis process) and/or analyzing a specimen material in accordance with certain embodiments of the invention. The process in FIG. 2 can include configuring a mold (process portion 202), providing a specimen material (process portion 204), breaking down/separating the specimen material (process portion 206), combining an embedment material with the specimen material (process portion 208), placing the specimen material into a mold (process portion 210), positioning material(s) in the mold (process portion 212), forming a specimen (process portion 214), removing the specimen from the mold (process portion 216), preparing the specimen (process portion 218), and analyzing at least a portion of the specimen (process portion 220).

In selected embodiments at least portions of the process in FIG. 2 can be used produce specimens efficiently and/or to produce specimens from specimen material that would otherwise be difficult to form into usable specimens. For example, in certain embodiments nanoparticulate materials can be embedded within an encapsulate material or embedment material and a specimen can be formed via casting the specimen using a mold. In selected embodiments, the embedment material can include a polymer, prepolymer, monomer, melt, eutectic, or other material. In certain embodiments, after casting, the formed specimen can be prepared for analysis (process portion 218) in a microanalysis process (e.g., cleaned, polished; sharpened) and then analyzed (process portion 220) using the microanalysis process. In other embodiments, the specimen does not require further processing after being formed in the mold. In still other embodiments, the specimen material can be non-nanoparticulate material or a single portion of bulk material. In still other embodiments, the specimen material can be placed in the mold without an embedment material.

In certain embodiments, configuring a mold (process portion 202) can include configuring a mold to form the specimen material into a shape suitable for a microanalysis process. In selected embodiments, the mold can be formed by forming mold material around at least a portion of an exemplar specimen shape and/or removing a portion of mold material from a structure of mold material. For example, in certain embodiments molds can be prepared from or using specimen or a specimen shape suitable for AP analysis (e.g., a conventional needle shape specimen several millimeters long, a microtip specimen that is tens of microns long, or a microtip array that includes multiple microtips). Information regarding desirable AP specimen shapes can be found in Kelly, T. F., P. P. Camus, et al. (1995), High Mass Resolution Local-Electrode Atom probe, USA, Wisconsin Alumni Research Foundation, U.S. Pat. No. 5,440,124; Kelly, T. F., R. L. Martens, et al. (2003), Methods of Sampling Specimens for Microanalysis, U.S. Pat. No. 6,700,121; and Kelly, T. F., J. J. McCarthy, et al. (1991), High Repetition Rate Position Sensitive Atom Probe, USA, Wisconsin Alumni Research Foundation, U.S. Pat. No. 5,061,850; Method to Determine 3-D Elemental Composition and Structure of Biological and Organic Material via Atom Probe Microscopy, WO2005/026684, filed Aug. 6. 2004, each of which is fully incorporated herein by reference.

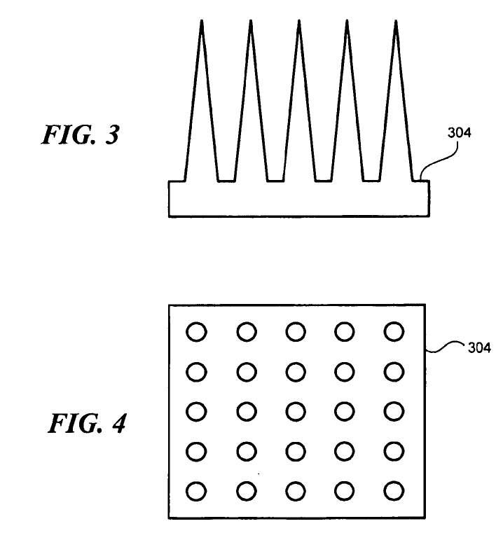

FIG. 3 is a partially schematic side view illustration of a microtip array 304 having a shape that is suitable for analysis in an AP in accordance with selected embodiments of the invention. FIG. 4 is a partially schematic top view illustration of the microtip array 304 shown in FIG. 3. In the illustrated embodiment, the microtip array can be formed using any of several microfabrication methods, such as those known for the production of silicon Micro-Electro-Mechanical Machines (“MEMS”). The microtip array can be created with the proper geometry of AP specimens. For example, in certain embodiments, the microtip array can be prepared from silicon using a reactive ion etching process, and can have a geometry where needle shape protuberances will stand proud about 50-100 um tall from a planer substrate. Each protuberance can have an end radius of 50-100 nm, and can be spaced about 100 microns to about 1 millimeter apart in a regular pattern.

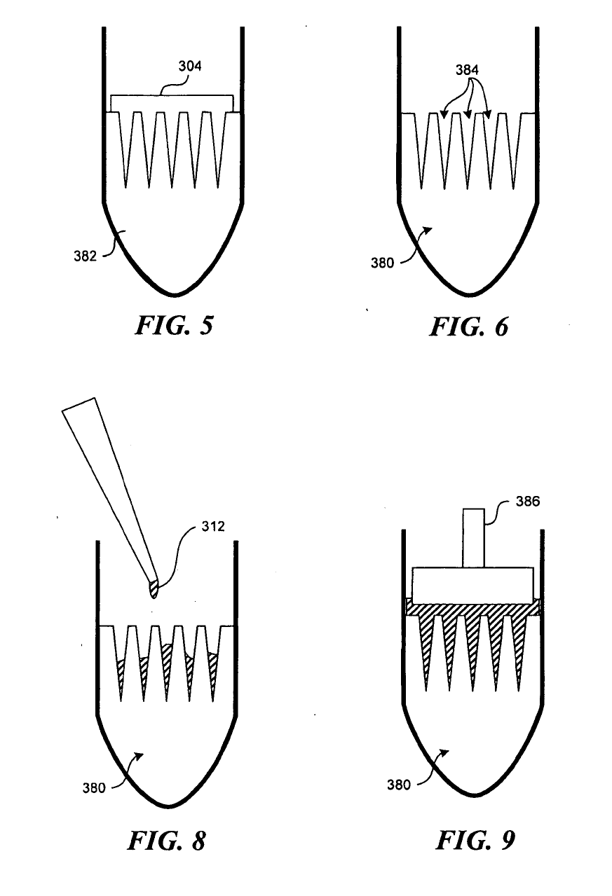

In FIG. 5, the microtip array 304 has been pressed into a mold material 382 (e.g., contained in a vessel) in accordance with certain embodiments of the invention. In the illustrated embodiment, the mold material includes a silicone rubber. The silicone rubber is then cured or polymerized and the microtip array is then removed. Accordingly, as shown in FIG. 6, a mold 380 having an array of voids 384 or void volumes is formed. The voids can have the proper net shape of the microtip atom probe needles so that the mold 380 can form specimens having the proper size, shape, and geometry for the associated microanalysis process.

In other embodiments, the mold material can include other materials that can be formed around an object to create a mold. For example, in selected embodiments the mold material can include polymers, prepolymers, metals, plastics, composites, ceramics, wax, and the like. In other embodiments, instead of a microtip array another suitable exemplar shape can be used to from a mold configured to form suitable specimens for various microanalysis processes.

In still other embodiments, a mold can be prepared by inserting electro-polished metal needle(s) (or similarly shaped long needles prepared from other materials) into a silicone rubber prepolymer (or other molding material) poured into a suitable vessel (such as a centrifuge tube). Once the silicone rubber is polymerized, the metal needles can be removed from the silicone, thereby leaving behind needle-shaped void(s) that can be used to cast specimen(s). In still other embodiments, a longer (e.g., circa centimeter long atom probe needles) can be used to prepare the molds.

As discussed above, yet other embodiments include forming a mold out of a structure of mold material, for example, using microfabrication to remove mold material from the structure. For example, in selected embodiments voids (e.g., with sub-micron resolution) can be formed in silicon or silicon oxide. The voids can be created by either abrasion (as with a diamond saw or laser ablation), etching, milling or some other method. Chemical etching can be accomplished by a number of methods such as using standard lithographic techniques including wet (as with KOH), dry (as with F) or plasma assisted (as with SFx) etching. Milling can be accomplished with a focused ion beam (FIB) or a broad ion beam with a masking arrangement to mask the areas where material removal is not desired. Other micromanufacturing methods can include, but are not limited to, a polymer based photoresist technique, wherein a solvent can be used to remove the photo-resist while keeping the polymer intact. Additional embodiments include direct photo-ablation processes and where acid-forming dyes are activated with photo-activation processes to locally create voids without the need for additional solvents.

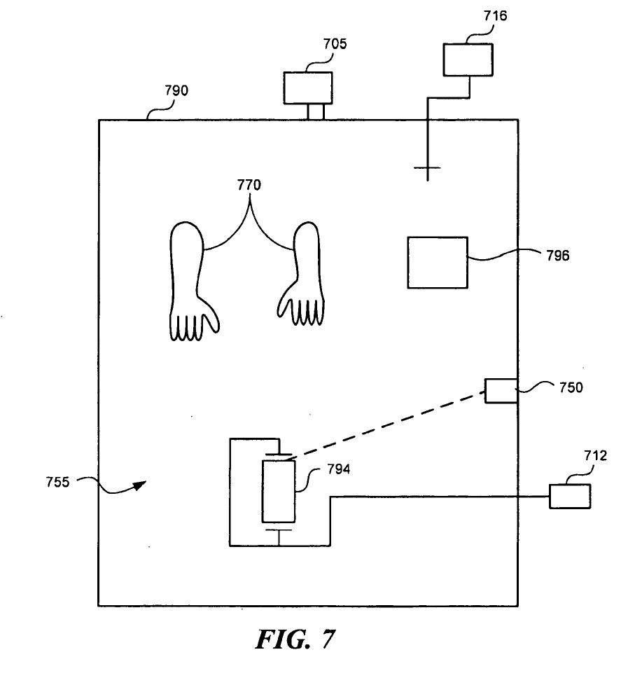

FIG. 7 is a partially schematic illustration of a processing arrangement 790 that can be suitable for carrying out various embodiments of the invention, including configuring molds and forming specimens. For example, one or more of the microfabrication processes used to remove mold material from a structure of mold material can be performed in a processing arrangement similar to the one shown in FIG. 7 (e.g., having some of the features shown in FIG. 7). The processing arrangement 790 in FIG. 7 can include an environmentally controlled chamber, container, or room. In the illustrated embodiment, the processing arrangement 790 includes a glove box having integral gloves 770, a fluid control device 705, an emitting device 750. The fluid control device 705 controls the pressure in the processing arrangement 790 and can introduce various fluids 755 (e.g., liquids or gases, including vapors or plasmas) into the container. The emitting device 750 can include various types of devices including an emitting device 750 that is configured to emit laser or photonic energy, radio frequency energy, an electron beam, a molecular beam, and/or an ion beam (including a focused ion beam and/or a broad ion beam). The processing arrangement 790 also includes a thermal control device 716 for controlling the temperature in the processing arrangement 790. Additionally, the processing arrangement 790 can include other devices 796 (e.g., mechanical devices, robotic arms, plunger devices, presses, grinders, saws, and the like).

In the illustrated embodiment, an item 794 is positioned in the processing arrangement 790. An energy source 712 (e.g., electrical source) can be provided so that it can create an electrical characteristic (e.g., an electrical field) proximate to the item 794 and/or apply an electrical characteristic (e.g., an electrical current) to the item 794. In some embodiments, the item 794 can include a block of mold material for forming a mold, specimen material (with or without an embedment material) for forming a specimen, a mold containing specimen material, or the like. Additionally, the processing arrangement 790 can include other devices 796 (e.g., mechanical devices, robotic arms, plunger devices, presses, grinders, saws, centrifuges, and the like) used in processing the item 794. For example, as discussed above, in certain embodiments mold material can be removed from a structure of mold material to form a mold. In other embodiments, the processing arrangement 790 can include more, fewer, and/or other arrangements of components.

Once a mold is configured to form material into a shape suitable for a microanalysis process, a specimen material can be provided (process portion 204) for microanalysis and placed in the mold (process portion 210). As discussed above, in selected embodiments the specimen material can be broken down and/or separated into separate portions (process portion 206) before being placed in the mold, Additionally, in selected embodiments an embedment material can be combined with the specimen material (process portion 208) before or after the specimen material is placed in the mold.

For example, the mold (e.g., the voids in the mold) can be filled with a specimen material or an embedding material that contains nanoparticles or broken down portions of specimen material. The specimen material or the specimen and embedment material can be positioned in the mold (process portion 212) using centrifugation, plunging, vacuum, and/or by other methods to, for example, force the material(s) to fill the mold appropriately. In selected embodiments, an electrical current characteristic can be used to position at least a portion of the specimen material and/or the embedment material. For example, in some embodiments an electric field can cause a migration of specimen material particles to migrate through an embedment material. In other embodiments, an electrical, magnetic, and/or optical field characteristic can be used to cause particles in the specimen materials and/or the embedment material to assume a selected orientation in the mold (e.g., to assume a selected alignment). Some or all of the processes described with respect to positioning material(s) in the mold can be carried out in a processing arrangement have features similar to those of the processing arrangement discussed above with reference to FIG. 7.

Once the material(s) has filled the voids it can then be solidified or hardened to form a specimen (process portion 214) suitable for use in a microanalysis process. For example, in selected embodiments, the specimen material or specimen and embedment materials can be hardened by polymerization (e.g., via annealing or the application of an electrical characteristic), cross-linking, cooling from a melt, heating or baking, a pressure application (e.g., from a plunger, press, or ambient pressure in a processing arrangement), a chemical agent (e.g. a catalyst), via photoactivation, and the like. In selected embodiments where the specimen is formed from a specimen material and an embedment material, the process of forming the specimen can cause the specimen and embedment material to bind together (e.g., stick together, bond together, or the like). Some or all of the processes described with respect to forming a specimen can be carried out in a processing arrangement have features similar to those of the processing arrangement discussed above with reference to FIG. 7.

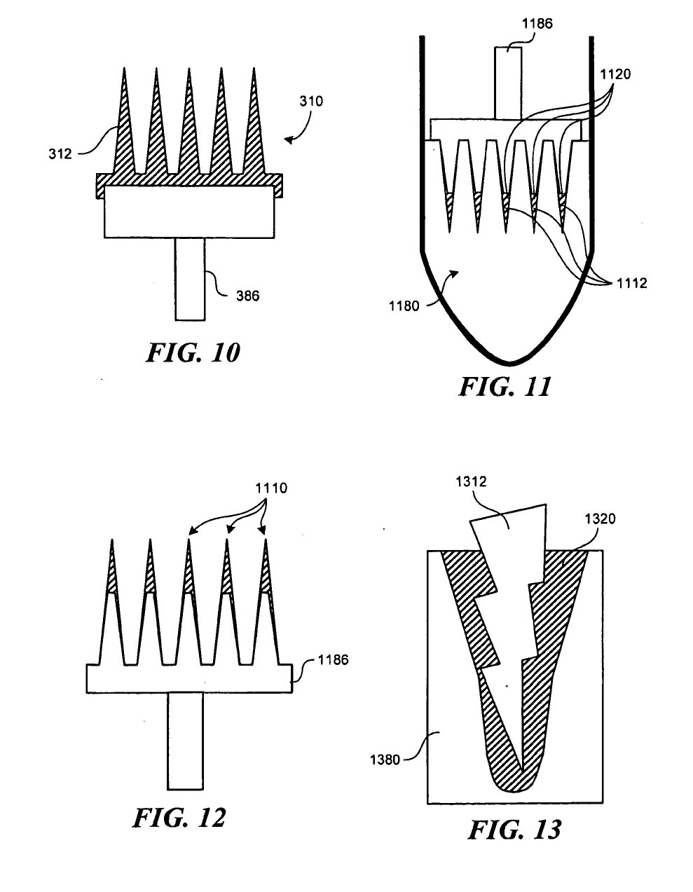

For example, FIG. 8 is a partially schematic illustration of a mold 380 being filled with a specimen material 312 in accordance with selected embodiments of the invention. In the illustrated embodiment, the specimen material 312 can be a liquid or a solid (e.g., a powder, chunks of material, or the like). FIG. 9 is a partially schematic illustration of the specimen material 312 being positioned in the mold 380 (shown in FIG. 8) by a plunger 386 in accordance with certain embodiments of the invention. FIG. 10 is a partially schematic illustration of a specimen 310 that includes the specimen material 312 after it has been removed from the mold shown in FIGS. 8 and 9 (e.g., after process portion 216) using the plunger.

In the illustrated embodiment, the plunger 386 can be used as a holder to retrieve the specimen and to support the specimen during subsequent handling, processing, and analysis. For example, in selected embodiments the plunger 386 can be electrically conductive and serve as a specimen holder during an AP process (e.g., transmitting an electrical potential to the specimen during AP analysis). In some embodiments, the plunger can also be thermally conductive and facilitate heating or cooling of the specimen, either during processing or analysis. In certain embodiments, the plunger 386 can receive various treatments prior to being used to form and/or remove the specimen. For example, these treatments can include mechanical treatments (e.g., roughening a surface of the plunger) or chemical treatments to improve adhesion, enhance electrical and/or thermal conductivity or provide other properties to improve specimen manipulations and/or analysis.

As shown in FIGS. 11 and 12, in other embodiments the plunger 1186 can have other shapes. For example, in FIGS. 11 and 12 the plunger 1186 resembles a microtip array, but with somewhat shorter and/or smaller microtips. In FIG. 11, the plunger 1186 is forcing a liquid that includes both a specimen material 1112 and an embedment material 1120 into a mold 1180. For example, in certain embodiments, the specimen material can be in solution with the embedment material, a suspended material in the embedment material, and/or a dispersed material in the embedment material. Although the mold 1180 is appropriately filled, the liquid only partially fills each void. Accordingly, after the liquid hardens or is set, the plunger 1186 can be used to remove and support multiple specimens from the mold, as shown in FIG. 12. In selected embodiments, the plunger 1186 can provide increased mechanical support to each of the specimens. In other embodiments, the exemplar shape used to configure the mold (e.g., the microtip array shown in FIG. 3-5) can be used as the plunger for the mold that was configured using the exemplar shape. For example, although the shape can be fully inserted in the mold material to form the mold, when acting as the plunger the shape is only partially inserted, forcing material(s) to the bottom of the mold, but leaving space for the specimen(s) to form.

Although in selected embodiments discussed above, the specimens are removed from the mold using a plunger, in other embodiments specimens are removed using other methods. For example, in certain embodiments a specimen can be removed from a mold by melting or other processes that destroy the mold whilst leaving the specimen intact (e.g., as in lost wax casting). This approach may be desirable with certain types of specimen materials and/or embedment materials, such as those that are particularly fragile, and when certain specimen geometries are required that cannot be readily removed from a mold. In some cases, as discussed above, additional processing or preparation of the specimen(s) (process portion 218) maybe accomplished prior to analysis (e.g., to enhance the analysis process).

In other embodiments, portions of an embedment material (e.g. nanoparticles) can be added to a suspension of spheres of indium alloy in a liquid flux poured into a mold with the proper shape for a microanalysis process. Following the addition of nanoparticles, heating can be used to melt the alloy and drives off the flux. The casting or specimen(s) can then removed from the mold. In other embodiments, nanoparticles may also be embedded within a polymer melt or by monomer/prepolymer polymerization using essentially the same protocol.

In still other embodiments, as shown in FIG. 13 a single piece of specimen material 1312 (e.g., a fiber, filament, wire, particle, piece, or the like) can be combined with and/or imbedded in an embedment material 1320 to produce a specimen that has the size, shape, and/or geometry suitable for a microanalysis process. In the illustrated embodiment, the single piece of specimen material 1312 can be placed in the mold 1380. An embedment material 1320 (e.g., a polymer, metal eutectic, or the like) can also be placed in the mold 1380. The embedment material can then be solidified or polymerized to form a specimen.

As discussed below in further detail, embedment materials can have additional features that can enhance the analysis process (e.g., thermal conductive properties which can be well suited for AP analysis using laser pulsing, evaporation characteristics, and the like). In some embodiments, a specimen material can be combined with an embedment material solely to receive an analysis enhancing feature.

For example, in selected embodiments involving AP analysis, image aberrations can be reduced in some circumstances by making a specimen at least approximately hemispherical in shape with at least approximately a smooth surface (e.g., with few or no voids, albeit with atomic-scale roughness). Additionally, it is sometimes desirable to maintain this configuration during field evaporation throughout the analysis. In selected embodiments where a specimen includes an embedment material, the characteristics of the embedment material can affect the surface condition of a specimen as a specimen is analyzed using a microanalysis process.

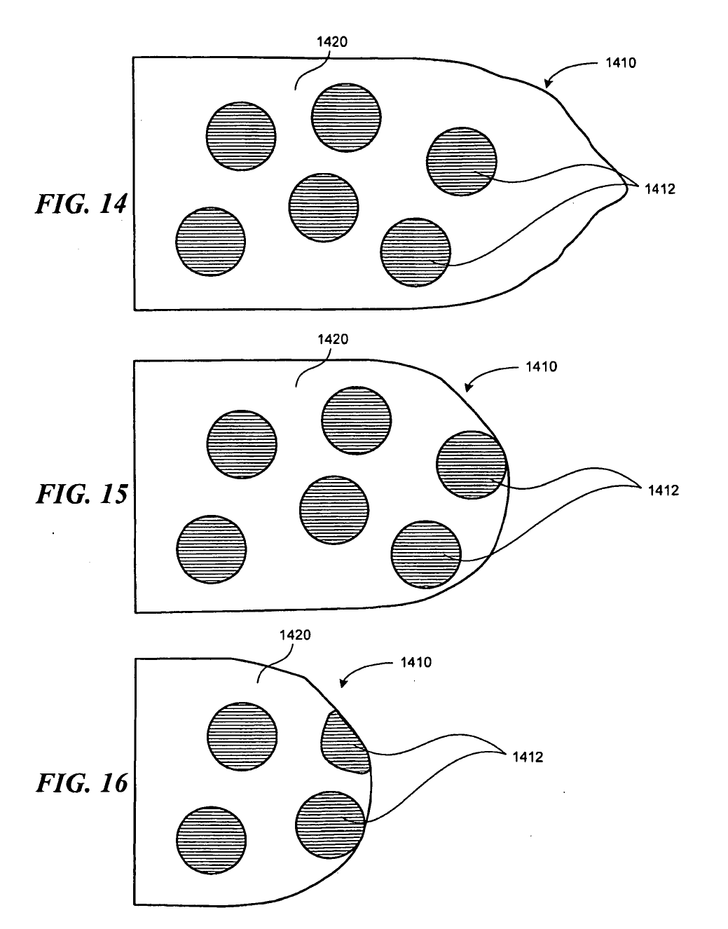

FIG. 14 shows a specimen 1410 suitable for an AP process that includes a specimen material 1412 combined with an embedment material 1420 prior to initiating AP analysis. In FIG. 14, the specimen material 1412 includes multiple noncontiguous portions spaced apart in an embedment material 1420. In the illustrated embodiment, the embedment material 1420 includes FE characteristics such that once the AP analysis process begins the “high points” of the embedment material will evaporate leaving a smoother more hemispherical type specimen (shown in FIG. 15). In the illustrated embodiment, the embedment material FE characteristics also are compatible with the specimen material FE characteristics so that as the AP analysis process continues, the overall tip shape remains at least approximately smooth and at least approximately hemispherical in shape as portions of the specimen material 1412 and portions of the embedment material 1420 are exposed near the tip of the specimen (as shown in FIG. 16).

In selected embodiments where embedment materials are included in the specimen, the analysis process (process portion 220) includes reconciling the date to account for the embedment material. In some embodiments, this can be accomplished using a computing system, such as the one shown in FIG. 1. For example, during an AP process the embedment material can be identified and accounted for or “removed” from the images produced so that the specimen material can be appropriately analyzed. In selected embodiments, an embedment material can be selected, at least in part, based on an identification characteristic that enables the embedment material to be readily analytically separated from the specimen material during data reduction.

Embedment materials can be chosen for any number of their characteristics. For example, these characteristics can include a selected thermal conductivity characteristic, a selected electrical conductivity characteristic, a selected work function characteristic, a selected erosion characteristic (e.g., evaporation characteristic), a selected identification characteristic (as discussed above), a selected compositional characteristic (e.g., elemental, isotopic, molecular, and/or structural) and/or a selected adhesive characteristic. In selected embodiments, the properties of the embedding matrix and how well it interfaces with the embedded nanoparticle can be important to successful imaging. For example, how well an embedment material binds (e.g., adhesive qualities) with a specimen material can be important. Additionally, if the specimen will be evaluated in an AP using pulse laser energy, the heat transfer characteristics (e.g., thermal conductivity characteristics) can be important because the laser energy produces thermal energy that aids in evaporation.

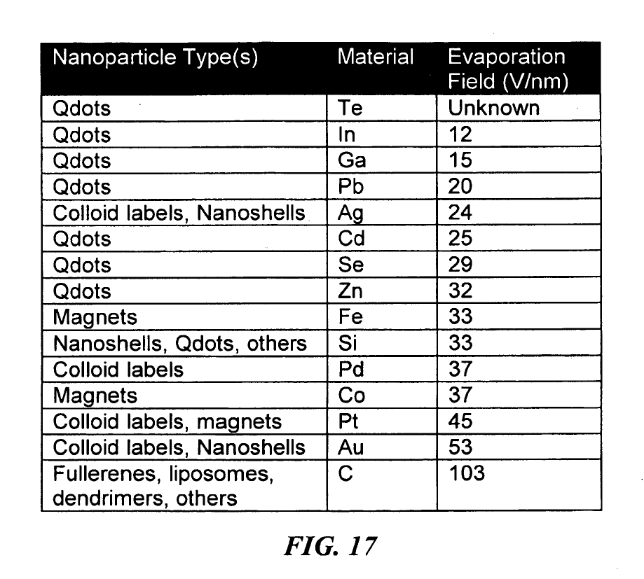

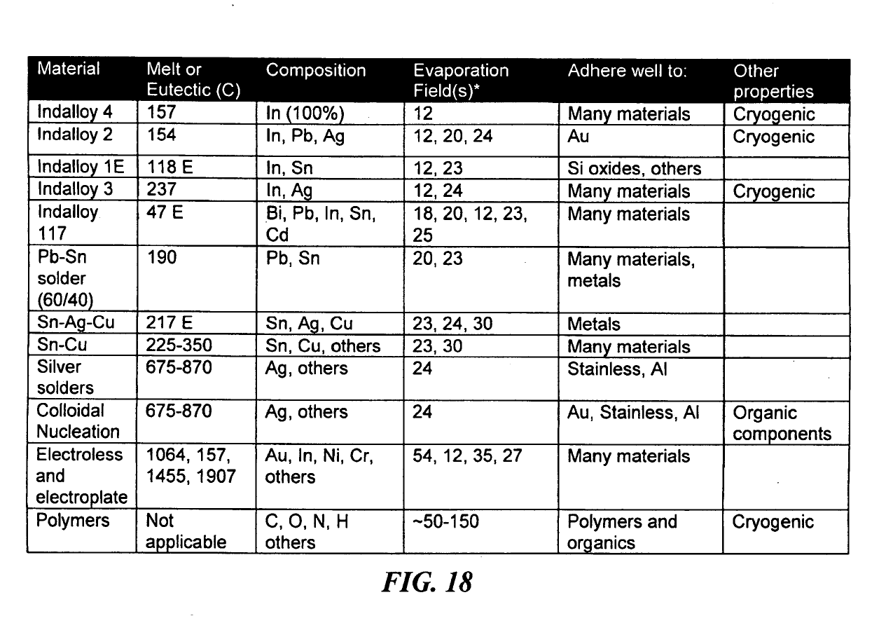

FE characteristics for certain types of materials are shown in FIG. 17. Based upon these characteristics it can be seen that a nanoparticle such as a gold (Au) colloid may require different embedment than Pd colloids since these materials field evaporate at different field strengths (53 vs. 37 V/nm). Similarly, if the Au colloid has a surface coating of Ag, then the properties of this coating also can be considered in the choice of embedment material and how the specimen will be prepared. Should the nanoparticle have additional components such as organics or ceramics, these components can also be considered. FIG. 18 provides information on other types of materials (e.g., materials that can be used as embedment materials). In various embodiments, one or more of the following properties or characteristics can be desirable for an embedment material:
Preserves and does not greatly alter the specimen material;
Binds well with the specimen material (e.g., adheres; covalently, ionically, or metallically bonds; or the like);
Holds specimen material immobilized and stable in electric field throughout analysis using an AP;
Adequate mechanical strength;
Able to be formed into a needle-like geometry with circa 100 nm tip radius for use in an AP;
Adequate electrical and/or thermal conductivity;
Field evaporates uniformly as single atoms or small molecular fragments while maintaining an at least approximately uniform, smooth, hemispherical surface throughout analysis when using an AP;
Field evaporates at the same (or similar) evaporation potential as the embedded specimen material when using an AP;
Includes a different elemental or isotopic composition from the specimen material to facilitate data reconciliation;
Convenient to prepare and combine with specimen material; and/or
Can encapsulate a sufficiently high particle density to place multiple particles in a given region of interest.

Polymers and/or conductive polymers (e.g., those that are inherently conductive or that have added material to make them conductive) have properties that make them useful for embedding specimen materials in selected embodiments of the invention. Some conductive polymers include, but are not limited to, polyanilines, polythiophenes, polyazines, polypyrroles and the like. In selected embodiments, the polymer may be processed into the molds as a prepolymer suspension, as a solution in a suitable solvent, as a melt, or as monomers that are polymerized in place within the mold. In certain embodiments, thermal annealing can be used to improve the physical properties of the embedment and to improve the binding between the polymer and embedded nanoparticles (e.g., for nanoparticles that will not be damaged by heating, including nanoparticles composed of metals or ceramics that can tolerate the temperatures associated with polymer annealing or melting). In selected embodiments, additional techniques can may be used to modulate either the electrical or thermal conductivity of a polymer. For example, in one embodiment small quantities of carbon nanotubes, carbon black, particulate metals, or other “dopants” can be added to the polymer to enhance the bulk electrical and/or thermal conductivity.

In yet other embodiments, low melting temperature metals and eutectics can be used as an embedment material. For example, in a selected embodiment a mold can be prepared from a silicone (e.g., such as Sylgard 184 available from Dow Corning). Because Sylgard and similar silicone rubbers can have continuous use temperatures of at least approximately 200° C., and short term stability to at least approximately 250° C., silicon molds can be suitable for casting specimens that use some indium alloys. In certain embodiments, some indium alloys can be obtained as particulate suspensions mixed with different fluxes to facilitate adhesion to a variety of materials including metals, oxides, and silicon. In other embodiments, Indiums and other low melting solders can be used as solids or powders. In still other embodiments, where higher melt/eutectic temperature materials are used, the molds can be produced from silicon, ceramics, and/or other materials. In selected embodiments, thermal annealing can be used to improve the physical properties of some metallic embedment materials and their binding properties with a specimen material (e.g., where the specimen material is tolerant of the associated heat).

In still other embodiments, electrical characteristics can be used to aid in binding certain embedment materials with certain specimen materials. For example, nanoparticles can be embedded in an embedment material during a casting process by filling the mold with the particles and the embedment material and applying an electrical characteristic (e.g., electrical current). The electrical characteristic can aid in binding the specimen material to the embedment material in a manner similar to the principles that apply to electroplating. In this way, the nanoparticles can be entrapped within the embedment material as the specimen forms within the mold. In certain embodiments, this process can be performed with Au, In, Ni, Cr and other materials.

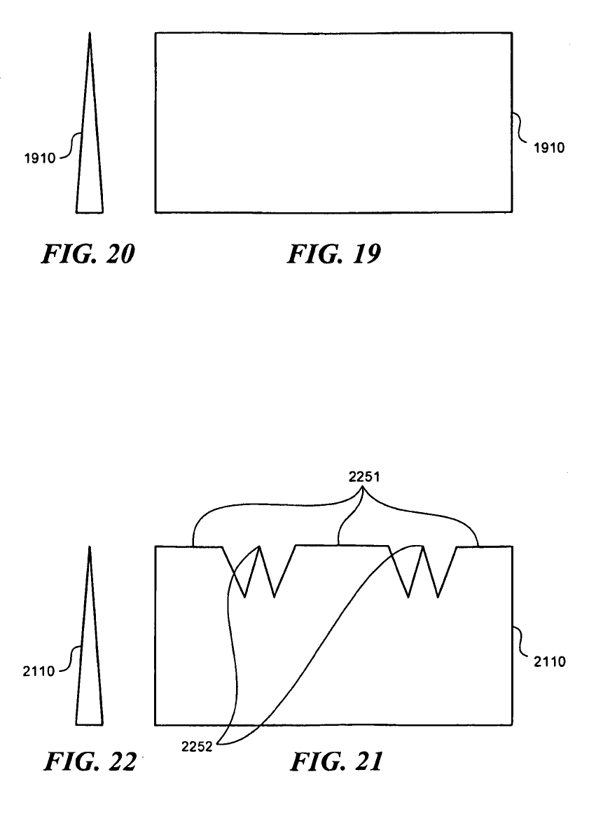

Although in many of the embodiments discussed above, the specimen have been formed into shapes, sizes, and/or geometries suitable for use in an atom probe (e.g., a needle shape or a microtip array), as discussed above, in other embodiments the specimen can include a shape, size, geometry, or other characteristic suitable for other types of microanalysis processes. For example, FIGS. 19 and 20 show a front view and a side view of a wedge shaped specimen 1910 suitable for use in a TEM process, a light microscopic process, an SPM process, and/or an AFM process since these processes provide wide fields of view with comparatively planar objects. In still other embodiments the specimen can have multiple parts, wherein different parts are suitable for different types of microanalysis processes, for example, to facilitate sequential analysis by multiple analytical and imaging instruments.

For example, FIGS. 21 and 22 illustrate a specimen 2110 that includes first parts 2251 and second parts 2252. The first parts 2251 can include a wedge type shape suitable for analysis in a TEM process and the second parts 2252 can include a needle or microtip shape suitable for use in an AP process. Accordingly, at least a portion of one or more of the first parts can be analyzed in a TEM and then at least a portion of one or more of the second parts can be analyzed in an AP. In selected embodiments, the first and second parts can be formed in a single mold or molding process. In other embodiments, a wedge shaped specimen can be formed in a mold and another process can be used to divide the specimen into first and second parts. For example, a focused ion beam can be used to cut portions of the wedge shape into needles. In still other embodiments, the first and second portions of the specimen can be formed without the use of a mold. For example, the first and second portions can be formed (e.g., using a focused ion beam) from a structure of material that includes a specimen material (e.g., a specimen material alone or a specimen material with an embedment material).

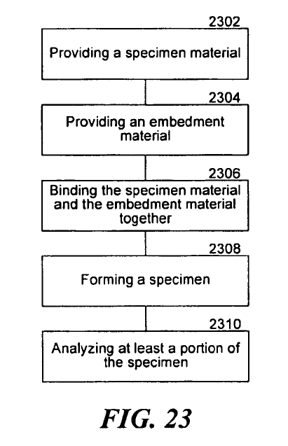

Although many of the embodiments discussed above have been discussed with reference to using a mold to form a specimen, in other embodiments many or all of the same features may be used and the specimen can be formed without using a mold. For example, as shown in FIG. 23, a process for producing a specimen and/or analyzing a specimen material can include providing a specimen material (process portion 2302), providing an embedment material (process portion 2304), and binding the specimen material and the embedment material together (process portion 2306). In certain embodiments, as discussed above, the specimen material can include multiple noncontiguous portions spaced apart from one another in the embedment material and/or the embedment material can have a selected characteristic (e.g., a selected thermal conductivity characteristic). The process can further include forming a specimen (process portion 2308). Although, as discussed above, in some embodiments, forming a specimen can be done via a mold, in other embodiments the specimen can be formed by removing material from a structure formed from the bound together specimen material and the embedment material (e.g., using a focused ion beam or other microfabrication process(es)).

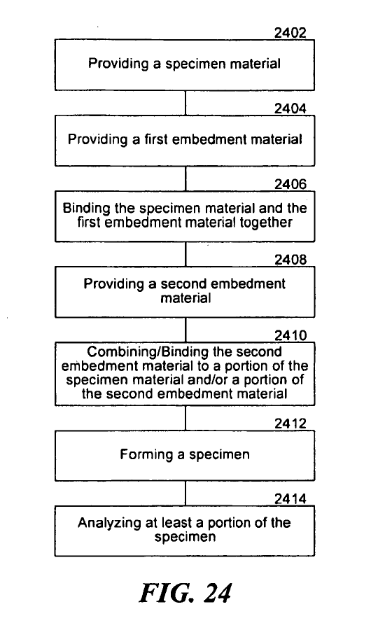

In still other embodiments, the process of using an embedment material can have multiple stages. For example, as shown in FIG. 24, in other embodiments a process for producing a specimen and/or analyzing a specimen material can include providing a specimen material (process portion 2402), providing a first embedment material (process portion 2404), and binding the specimen material and the first embedment material together (process portion 2406). The process can further include providing a second embedment material (process portion 2408), and combining/binding the second embedment material to a portion of the specimen material and/or a portion of the second embedment material (process portion 2410). For example, in selected embodiments the bound together specimen material and first embedment material can be placed in a mold and combined/bound with the second embedment material. In other embodiments the bound together specimen material and first embedment material combined/bound with the second embedment material without using a mold (e.g., to form a structure from which material can be removed to form a specimen). The process can still further include forming a specimen (process portion 2412) and analyzing at least a portion of the specimen (process portion 2414).

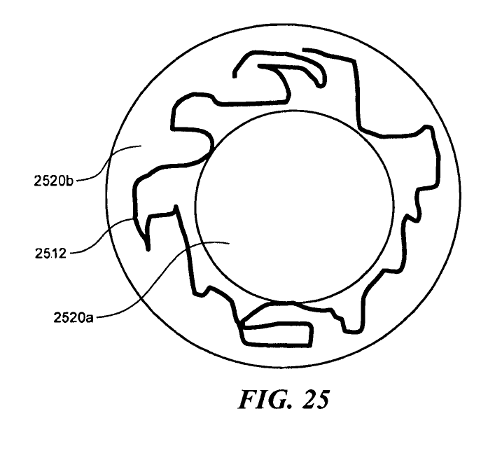

In selected embodiments, the process discussed above with reference to FIG. 24, can be particularly useful for analyzing biological materials such as amino acids and/or proteins. In other embodiments, the process can be useful for analyzing various polymers. For example, as shown in FIG. 25 a specimen Material 2512 (e.g., an amino acid, protein, polymer, or the like) can be bound to a first embedment material 2520a. For example, in certain embodiments the specimen material can be bound to or around gold (e.g., as in a colloidal nucleation process), carbon nanotubes, buckyballs, quantum dots, dendrimers, cadmium sulfide, cadmium selenide (nanoparticles), paladium, aluminum, and the like. A second embedment material 2520b (e.g., silver) can then be combined with or bound to a portion of the specimen material and/or a portion of the second embedment material. A specimen can then be formed and the material analyzed. As discussed above, analyzing the data can include reconciling the data to account for the first and/or second embedment materials.

From the foregoing, it will be appreciated that specific embodiments of the invention have been described herein for purposes of illustration, but that various modifications may be made without deviating from the invention. Additionally, aspects of the invention described in the context of particular embodiments may be combined or eliminated in other embodiments. Although advantages associated with certain embodiments of the invention have been described in the context of those embodiments, other embodiments may also exhibit such advantages. Additionally, not all embodiments need necessarily exhibit such advantages to fall within the scope of the invention. Accordingly, the invention is not limited except as by the appended claims.

I/We claim:

1. A method for producing a specimen for a microanalysis process, comprising:
providing material to be analyzed via a microanalysis process,
placing the material into a mold configured to form a specimen suitable for the microanalysis process; and
forming a specimen suitable for use in the microanalysis process using the mold, the specimen including the material.

2. The method of claim 1 wherein the method further comprises breaking down the material before placing the material into the mold.

3. The method of claim 1 wherein the method further comprises at least one of cutting the material into pieces, grinding the material, dissolving the material, and melting the material before placing the material into the mold.

4. The method of claim 1 wherein the microanalysis process includes at least one of an atom probe process, a transmission electron microscopy process, a mass spectrometer process, a diffraction process, and a matrix-assisted laser desorption/ionization process.

5. The method of claim 1 wherein forming a specimen includes applying pressure to at least a portion of the material.

6. The method of claim 1 wherein forming a specimen includes cooling the at least a portion of the material placed.

7. The method of claim 1 wherein forming a specimen includes forming a specimen having a first part suitable for use in a first microanalysis process and a second part suitable for use in a second microanalysis process.

8. The method of claim 1 wherein forming a specimen suitable for use in the microanalysis process includes forming the material into at least one of a wedge suitable for use in a transmission electron microscopy process, a microtip array suitable for use in an atom probe process, and a needle shape suitable for use in an atom probe process.

9. The method of claim 1 wherein the method further comprises positioning at least a portion of the material in the mold using a plunger assembly.

10. The method of claim 1 wherein the method further comprises:
positioning at least a portion of the material in the mold using a plunger assembly; and
removing the specimen from the mold using the plunger assembly.

11. The method of claim 1 wherein the method further comprises positioning at least a portion of the material in the mold using centrifugal force.

12. The method of claim 1 wherein the method further comprises configuring a mold to form the material into a shape suitable for the microanalysis process.

13. The method of claim 1 wherein the method further comprises configuring a mold to form the material into a shape suitable for the microanalysis process, wherein configuring a mold includes forming mold material around at least a portion of an exemplar specimen shape and fabricating a mold by removing a portion of mold material from a structure of mold material.

14. The method of claim 1 wherein the material includes a specimen material and wherein the method further comprises combining an embedment material with the specimen material prior to forming the specimen.

15. The method of claim 1 wherein the material includes a specimen material and wherein the method further comprises combining an embedment material with the specimen material prior to forming the specimen, the embedment material having at least one of a selected thermal conductivity characteristic, a selected electrical conductivity characteristic, a selected work function characteristic, a selected erosion characteristic, a selected compositional characteristic, and a selected adhesive characteristic.

16. The method of claim 1 wherein the material includes a specimen material and wherein the method further comprises combining an embedment material with the specimen material prior to forming the specimen, the specimen material including multiple noncontiguous portions spaced apart in the embedment material.

17. The method of claim 1 wherein the material includes a specimen material and wherein the method further comprises combining an embedment material with the specimen material prior to forming the specimen, the embedment material including a polymer.

18. The method of claim 1 wherein the material includes a specimen material and wherein the method further comprises combining an embedment material with the specimen material prior to forming the specimen, the embedment material including a polymer, and further wherein forming the specimen includes at least one of annealing the polymer and electrically polymerizing the polymer.

19. The method of claim 1 wherein the material includes a specimen material and wherein the method further comprises:
combining an embedment material with the specimen material prior to forming the specimen; and
using an electrical current characteristic to position at least a portion of (a) the specimen material, (b) the embedment material, or (c) both (a) and (b) in the mold.

20. The method of claim 1 wherein the method further comprises preparing the specimen for the microanalysis process after the specimen has been formed.

21. The method of claim 1 wherein the material includes a specimen material and wherein the method further comprises combining an embedment material with the specimen material prior to forming the specimen, and further wherein forming a specimen includes using at least one of a chemical process and an electrical current characteristic to aid in binding the specimen material and the embedment material together.

22. The method of claim 1 wherein the material includes a specimen material and wherein the method further comprises:
binding a first embedment material to the specimen material prior to placing the specimen material into the mold; and
combining a second embedment material with at least one of a portion of the specimen material and a portion of the first embedment material prior to forming the specimen.

23. A method for analyzing a specimen material using a microanalysis process, comprising:
providing specimen material to be analyzed via a microanalysis process,
placing the specimen material into a mold configured to form the specimen material into a shape suitable for the microanalysis process;
forming a specimen suitable for use in the microanalysis process using the mold, the specimen including the specimen material; and
analyzing at least a portion of the specimen using the microanalysis process.

24. The method of claim 23 wherein the method further comprises:
positioning the material in the mold using a plunger assembly; and
removing the specimen from the mold using the plunger assembly, wherein analyzing at least a portion of the specimen includes analyzing at least a portion of the specimen while the specimen is coupled to at least a portion of the plunger assembly.

25. The method of claim 23 wherein forming a specimen includes forming a specimen having a first part suitable for use in a first microanalysis process and a second part suitable for use in a second microanalysis process, and where analyzing at least a portion of the specimen includes analyzing at least a portion of the first part of the specimen using the first microanalysis process and analyzing at least a portion of the second part of the specimen using the second microanalysis process.

26. The method of claim 23 wherein the method further comprises combining an embedment material with the specimen material prior to forming the specimen and wherein analyzing at least a portion of the specimen includes reconciling the data to account for the embedment material.

27. A method for producing a specimen for a microanalysis process, comprising:
providing a specimen material to be analyzed via a microanalysis process;
providing an embedment material;
binding the specimen material and the embedment material together, the specimen material including multiple noncontiguous portions spaced apart from one another in the embedment material; and
forming a specimen from the specimen material and the embedment material that are bound together, the specimen including the multiple noncontiguous portions spaced apart from one another in the embedment material.

28. The method of claim 27 wherein the microanalysis process includes at least one of an atom probe process, a transmission electron microscopy process, a mass spectrometer process, a diffraction process, and a matrix-assisted laser desorption/ionization process.

29. The method of claim 27 wherein forming a specimen includes forming a specimen via at least one of a casting process and a material removal process.

30. The method of claim 27 wherein forming a specimen includes forming a specimen having a first part suitable for use in a first microanalysis process and a second part suitable for use in a second microanalysis process.

31. The method of claim 27 wherein providing an embedment material includes providing an embedment material having at least one of a selected thermal conductivity characteristic, a selected electrical conductivity characteristic, a selected work function characteristic, a selected erosion characteristic, a selected compositional characteristic, and a selected adhesive characteristic.

32. The method of claim 27 wherein providing an embedment material includes providing a first embedment material and wherein the method further includes binding a second embedment material to at least one of a portion of the first embedment material and a portion the specimen material prior to forming the specimen.

33. A method for analyzing a specimen material using a microanalysis process, comprising:
providing a specimen material to be analyzed via a microanalysis process;
providing an embedment material;
binding the specimen material and the embedment material together, the specimen material including multiple noncontiguous portions spaced apart from one another in the embedment material;
forming a specimen from the specimen material and the embedment material that are bound together, the specimen including the multiple noncontiguous portions spaced apart from one another in the embedment material; and
analyzing at least a portion of the specimen using the microanalysis process.

34. The method of claim 33 wherein forming a specimen includes forming a specimen having a first part suitable for use in a first microanalysis process and a second part suitable for use in a second microanalysis process, and where analyzing at least a portion of the specimen includes analyzing at least a portion of the first part of the specimen using the first microanalysis process and analyzing at least a portion of the second part of the specimen using the second microanalysis process.

35. The method of claim 33 wherein analyzing at least a portion of the specimen includes reconciling the data to account for the embedment material.

36. A method for producing a specimen for a microanalysis processes, comprising:
providing a specimen material to be analyzed via a microanalysis process;
providing an embedment material;
binding the specimen material and the embedment material together, the embedment material having a selected thermal conductivity characteristic; and
forming a specimen from the specimen material and the embedment material that are bound together.

37. The method of claim 36 wherein the microanalysis process includes at least one of an atom probe process, a transmission electron microscopy process, a mass spectrometer process, and a matrix-assisted laser desorption/ionization process.

38. The method of claim 36 wherein forming a specimen includes forming a specimen via at least one of a casting process and a material removal process.

39. The method of claim 36 wherein forming a specimen includes forming a specimen having a first part suitable for use in a first microanalysis process and a second part suitable for use in a second microanalysis process.

40. The method of claim 36 wherein providing an embedment material includes providing an embedment material having at least one of a selected electrical conductivity characteristic, a selected work function characteristic, a selected erosion characteristic, and a selected adhesive characteristic.

41. A method for analyzing a specimen material using a microanalysis process, comprising:
providing a specimen material to be analyzed via a microanalysis process;
providing an embedment material;
binding the specimen material and the embedment material together, the embedment material having a selected thermal conductivity characteristic;
forming a specimen from the specimen material and the embedment material that are bound together; and
analyzing at least a portion of the specimen using the microanalysis process.

42. The method of claim 41 wherein forming a specimen includes forming a specimen having a first part suitable for use in a first microanalysis process and a second part suitable for use in a second microanalysis process, and where analyzing at least a portion of the specimen includes analyzing at least a portion of the first part of the specimen using the first microanalysis process and analyzing at least a portion of the second part of the specimen using the second microanalysis process.

43. The method of claim 41 wherein analyzing at least a portion of the specimen includes reconciling the data to account for the embedment material.

44. A method for producing a specimen for a microanalysis process, comprising:
providing a specimen material to be analyzed via a microanalysis process;
providing a first embedment material;
binding the specimen material and the first embedment material together;
providing a second embedment material;
binding the second embedment material to at least one of a portion of the specimen material and a portion of the second embedment material; and
forming a specimen from the specimen material, the first embedment material, and the second embedment material after the second embedment material is bound to the at least one of the portion of the specimen material and the portion of the second embedment material.

45. The method of claim 44 wherein the specimen material includes at least one of a protein, an amino acid, and a polymer.

46. The method of claim 44 wherein the specimen material includes at least one of a protein, an amino acid, and a polymer, the first embedment material includes gold and the second embedment material includes silver.

47. A method for analyzing a specimen material using a microanalysis process, comprising:
providing a specimen material to be analyzed via a microanalysis process;
providing a first embedment material;
binding the specimen material and the first embedment material together;
providing a second embedment material;
binding the second embedment material to at least one of a portion of the specimen material and a portion of the second embedment material;
forming a specimen from the specimen material, the first embedment material, and the second embedment material after the second embedment material is bound to the at least one of the portion of the specimen material and the portion of the second embedment material; and
analyzing at least a portion of the specimen using the microanalysis process.
